(19) United States
(12) Patent Application Publication
(10) Pub. No.: US 2007/0112216 A1
(43) Pub. Date: May 17, 2007

(54) Method for producing chiral mercapto amino acids

(76) Inventors: Martina Kotthaus, Linz (AT); Herbert Mayrhofer, Engerwitzdorf (AT); Christian Rogl, St. Marlen (AT); Sylvia Krich, Halbach (AT); Michael Simetzberger, Linz (AT)

Correspondence Address: NIXON & VANDERHYE, PC, 901 NORTH GLEBE ROAD, 11TH FLOOR, ARLINGTON, VA, 22203, US

(21) Appl. No.: 10/581,790

(22) PCT Filed: Nov. 15, 2004
(86) PCT No.: PCT/EP04/12919

§ 371 Date: Jun. 6, 2006

(30) Foreign Application Priority Data
Dec. 9, 2003 (AT) A 1968/2003

(51) Int. Cl. C07C 323/29

(52) U.S. Cl. 562/426; 562/556

(57) ABSTRACT

The invention relates to a method for producing chiral mercapto amino acids of formula (I) wherein R₁, R₂ and R₃ can represent hydrogen, C₆-C₁₂ aryl, C₁-C₆-alkyl-C₆-C₁₂-aryl, C₆-C₁₂-aryl-C₁-C₆-alkyl, C₁-C₁₈-alkyl or C₂-C₁₈-alkenyl, R₂ and R₃ forming a saturated or unsaturated ring. According to said method, a) an oxo compound of formula (II), wherein X represents a leaving group, is reacted in the presence of ammonia or ammonium hydroxide and a sulfide, optionally under phase transfer catalysis or addition of a solubiliser, with a ketone or an aldehyde of formula (III) wherein R₄ and R₅ can represent a C₁-C₁₂ alkyl radical or a C₆-C₂₀ aryl radical or one of the two radicals H, or R₄ and R₅ together form a C₄-C₇ ring, to form the compound of formula (IV), that b) reacts with HCN to form the corresponding nitrile, whereupon c) the crystallised nitrile is converted, by selective hydrolysis by means of a mineral acid, into the corresponding amide of formula (VI), and d) is then converted into the corresponding chiral amide of formula (VI*) by means of an L amidase or a chiral dissociating acid, whereupon by reaction with an acid, the desired chiral mercapto amino acid of formula (I) is obtained, or e) first the reaction with an acid is carried out, and then the conversion into the chiral mercapto amino acid takes place.

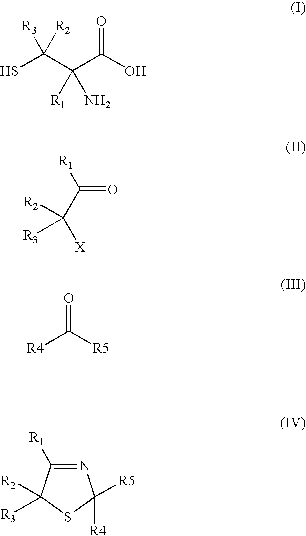

Chiral mercapto amino acids such as, for instance, alpha-methylcysteine or penicillamines are used for example as intermediates for preparing pharmaceuticals such as, for instance, iron chelators (S-alpha-methyl-cysteine, antirheumatic (R-alpha-methylcysteine) or as HIV protease inhibitor (L-penicillamine). Because of the strict regulations concerning cross-contamination with antibiotics, chemical synthetic routes for example for penicillamines, which can also be obtained at reasonable cost from Pen-G, are in great demand. The preparation of chiral mercapto amino acids, for example of (S)-alpha-methylcysteine, takes place for example in analogy to Tetrahedron 1993, 49 (24), 5359-5364 in a Seebach-analogous synthesis by acid hydrolysis of 2S,4S-methyl 2-tert-butyl-1,3-thiazolidine-3-formyl-4-methyl-4-carboxylate. 2S,4S-Methyl 2-tert-butyl-1,3-thiazolidine-3-formyl-4-methyl-4-carboxylate is in this case prepared starting from (S)-cysteine methyl ester and pivaldehyde via 2S-methyl 2-tert-butyl-1,3-thiazolidine-4-carboxylate, introduction of a formyl protective group to give 2S,4S-methyl 2-tert-butyl-1,3-thiazolidine-3-formyl-4-carboxylate, reaction at −78° C. with lithiumdiisopropyl-amide to give the corresponding enolate and quenching of the enolate with methyl iodide. The yield of (S)-α-methylcysteine starting from (S)-cysteine ethyl ester in this case is only 29%. Besides the low yield of (S)-α-methylcysteine, the substantial disadvantages of this preparation variant are the elaborate process steps and, in particular, the starting material (S)-cysteine methyl ester hydrochloride, of an unnatural compound which is not commercially available and is therefore not to be considered for industrial syntheses.

The preparation of racemic cysteine is disclosed for example in Angew. Chem. 93 (1981) No. 8, pp. 680 et seq., according to which DL-cysteine hydrochloride H₂O is obtained starting from chloroacetaldehyde, sodium bisulfite, ammonia and acetone via 2,2-dimethyl-3-thiazoline, subsequent reaction with anhydrous hydrocyanic acid to give 2,2-dimethylthiazolidin-4-carbonitrile and final addition of aqueous hydrochloric acid.

It was an object of the present invention to find a suitable process for preparing chiral mercapto amino acids which provides the desired final compounds in a simple and cost-effective manner in high yield and in high optical purity.

It has surprisingly been possible to achieve this object inter alia by selecting specific ketones as precursors.

The present invention accordingly relates to a process for preparing chiral mercapto amino acids of the formula 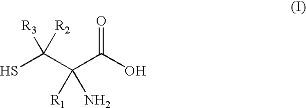
in which R₁, R₂ and R₃ may be identical or different and may be hydrogen, C₆-C₁₂-aryl, C₁-C₆-alkyl-C₆-C₁₂-aryl, C₆-C₁₂-aryl-C₁-C₆-alkyl, C₁-C₁₈-alkyl or C₂-C₁₈-alkenyl, where R₂ and R₃ may form a saturated or unsaturated ring, and the radicals may optionally be substituted one or more times by F, NO₂ or CN, which is characterized in that
a) an oxo compound of the formula 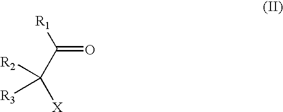
   in which R₁, R₂ and R₃ are as defined above, and X is a leaving group from the group of Cl, Br, iodine, triflate, acetate or of the sulfonates, is reacted in the presence of ammonia or ammonium hydroxide and of a sulfide from the group of ammonium hydrosulfide, alkaline earth metal hydrosulfides or alkali metal hydrosulfides, where appropriate with phase-transfer catalysis or with addition of a solubilizer, with a ketone or aldehyde of the formula 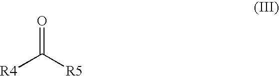
   in which R₄ and R₅ may be identical or different and may be a C₁-C₁₂-alkyl radical or a C₆-C₂₀-aryl radical or one of the two radicals may be H, or R₄ and R₅ together form a C₄-C₇ ring which may optionally be substituted one or more times by C₁-C₆-alkyl or C₆-C₂₀-aryl, to give the compound of the formula 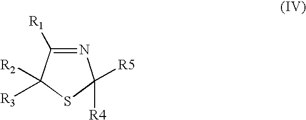
   in which R₁, R₂, R₃, R₄ and R₅ are as defined above, which
b) react with HCN to give the compound of the formula 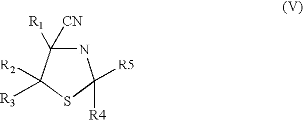
   in which R₁, R₂, R₃, R₄ and R₅ are as defined above, after which
c) the crystallized compound of the formula (V) is converted by selective hydrolysis using a mineral acid into the corresponding amide of the formula 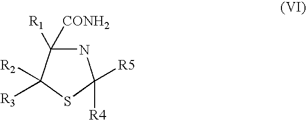
   in which R₁, R₂, R₃, R₄ and R₅ are as defined above, and
d) subsequently converted using an amidase or a chiral resolving acid into the corresponding chiral amide of the formula (VI*), after which the desired chiral mercapto amino acid of the formula (I) is obtained by reaction with an acid, or
e) firstly the reaction of the amide with an acid is carried out, and subsequently the conversion into the desired chiral mercapto amino acid of the formula (I) takes place.

Chiral mercapto amino acids of the formula (I) are prepared by the process of the invention.

R₁, R₂ and R₃, in the formula (I) may be identical or different and may be hydrogen, C₆-C₁₂-aryl, C₁-C₆-alkyl-C₆-C₁₂-aryl C₆-C₁₂-aryl-C₁-C₆-alkyl, C₁-C₁₈-alkyl or C₂-C₁₈-alkenyl.

C₁-C₁₈-Alkyl means in this connection linear, branched or cyclic alkyl radicals such as, for instance, methyl, ethyl, i-propyl, n-propyl, cyclopropyl, n-butyl, sec-butyl, tert-butyl, n-pentyl, n-hexyl, cyclohexyl, 2-ethylhexyl, n-octyl, cyclooctyl, n-dodecyl etc.

C₁-C₁₂-Alkyl radicals are preferred, and C₁-C₄-alkyl radicals are particularly preferred.

C₂-C₁₈-Alkenyl radicals means linear, branched or cyclic alkenyl radicals which have one or more double bonds, such as, for instance, ethylene, propenyl, 1-butenyl, isobutenyl, 2-pentenyl, 2-methyl-1-butenyl, propandienyl, cyclopentenyl, cyclohexenyl etc.

C₂-C₁₂-Alkenyl radicals are preferred, and C₂-C₆-alkenyl radicals are particularly preferred.

Examples of C₆-C₁₂-aryl radicals are phenyl, naphthyl, indenyl etc.

Preferred aryl radicals are phenyl and naphthyl, and the phenyl radical is particularly preferred.

Examples of C₁-C₆-alkyl-C₆-C₁₂-aryl radicals are p-tolyl, o-xylyl, 4-ethylphenyl, 4-tert-butylphenyl etc. In this connection, C₁-C₄-alkyl-C₆-aryl radicals are preferred, and C₁-C₂-alkylphenyl radicals are particularly preferred.

Examples of suitable C₆-C₁₂-aryl-C₁-C₆-alkyl radicals are phenylpropyl, benzyl, phenylethyl etc.

In this connection, C₆-aryl-C₁-C₄-alkyl radicals are preferred, particularly preferably phenyl-C₁-C₂-alkyl radicals.

R₂ and R₃ may, however, also together form a saturated or unsaturated ring which then preferably comprises from 3 to 12 C atoms and particularly preferably from 4 to 10 C atoms.

The radicals R₁, R₂ and R₃ may moreover be optionally substituted one or more times by F, NO₂ or CN.

Examples of compounds of the formula (I) which can be prepared according to the invention are alpha-methylcysteine, penicillamines, cysteine or beta-mercaptophenylalanine.

In the first step of the process of the invention, an oxo compound of the formula (II) is reacted with a ketone or aldehyde of the formula (III).

The radicals R₁, R₂ and R₃ in the formula (II) are as defined above, and X is a leaving group such as chlorine, bromine, iodine, triflate, acetate or a sulfonate such as, for instance, mesylate, tosylate or phenylsulfonate. X is preferably chlorine, bromine or iodine and particularly preferably chlorine.

Examples of suitable oxo compounds of the formula (II) are chloroacetaldehyde, chloroacetone, alpha-chloroiso-butyraldehyde, 2-chloropropanal, 2-chloro-n-butanal, 2-bromo-n-butanal or phenacyl bromide.

R₄ and R₅ in the formula (III) are independently of one another a C₁-C₁₂-alkyl radical, preferably a C₁-C₆-alkyl radical, or a C₆-C₁₂-aryl radical, preferably a phenyl radical, or one of the two radicals H.

R₄ and R₅ may, however, also together form a C₄-C₇ ring, preferably a C₅-C₆ ring, which may be substituted one or more times by C₁-C₆-alkyl, preferably by C₁-C₄-alkyl, or C₆-C₂₀-aryl, preferably by phenyl.

Cyclic ketones are preferred.

Examples of suitable ketones of the formula (II) are cyclohexanone, cyclopentanone, 2-methylcyclohexanone, diphenyl ketone, acetone, diethyl ketone.

The reaction takes place in the presence of ammonia or ammonium hydroxide and of a sulfide.

Suitable sulfides in this connection are ammonium hydrosulfide, alkaline earth metal hydrosulfides or alkali metal hydrosulfides. Sodium hydrosulfide or potassium hydrosulfide are preferably employed.

The ammonia or the ammonium hydroxide can be introduced as such or as solution.

Preferably from 1 to 5 mol of ketone or aldehyde, particularly preferably from 2 to 3.5 mol of ketone or aldehyde, are added per mole of oxo compound of the formula (II).

The sulfide compound is employed in an amount of from 1 to 3 mol per mole of oxo compound, preferably from 1.1 to 2 mol per mole of oxo compound.

The amount of added ammonia or ammonium hydroxide is from 1 to 5 mol, preferably 1.5 to 3.5 mol, per mol of oxo compound.

The reaction in this case can, if the ketone or aldehyde of the formula (III) serves as solvent, be carried out without additional solvent, or else take place in the presence of a solvent from the group of water, C₁-C₄-alcohols or of aromatic or aliphatic hydrocarbons which may optionally be halogenated, or in mixtures thereof.

The reaction is preferably carried out in a mixture of ketone/aldehyde of the formula (III) and water.

The sequence of addition can in principle be chosen without restriction, but the ketone or aldehyde and the sulfide compound are preferably introduced first, and then ammonia or ammonium hydroxide and the oxo compound are added.

The reaction temperature is in this case from −10° C. to +30° C., preferably from −5° C. to +15° C.

After all the reactants have been added, the reaction mixture is stirred for from 5 to 300 minutes, preferably for from 10 to 120 minutes and particularly preferably for from 20 to 60 minutes, at 0 to 70° C.

However, the reaction may also take place with phase-transfer catalysis or with addition of a solubilizer. Phase-transfer catalysts suitable for this purpose are tetrabutylammonium bromide, tetrabutylammonium chloride, tetrabutylammonium bisulfate, tetrabutyl-ammonium nitrate, tetrabutylammonium tetraphenylborate, benzyltributylammonium chloride, tributylmethylammonium bromide, triethylmethylammonium chloride, aliquot 336 (3-methyltrioctylammonium chloride), aliquot HTA-1, Adogen 464 (methyltrialkyl (C₈-C₁₀) ammonium chloride, sodium tetraphenylborate, ammonium tetraphenylborate etc.

The catalyst is in this case added in an amount of from 1 to 15 mol %, preferably from 3 to 8 mol %, based on oxo compound of the formula (II).

Examples of suitable solubilizers are acetonitrile, tetrahydrofuran, dimethylformamide, dioxane, pyridine, N-methylpyrrolidone etc.

The reaction temperature is once again from −10° C. to +30° C., preferably from −5° C. to +15° C.

The thiazoline compound of the formula (IV) obtained in this way is then isolated from the reaction mixture, for example by fractional distillation of the organic phase.

Subsequently, in step b), the thiazoline compound of the formula (IV) is reacted with HCN.

HCN can in this case be employed as such, gaseous or liquid or as solution in water or organic solvents or prepared as intermediate from NaCN and acid.

The amount of HCN employed is from 1 to 5 mol, preferably 1.5 to 3.5 mol, per mol of thiazoline compound.

The reaction in this case takes place in a solvent from the group of water, C₁-C₄ alcohol, ester, ether or of aliphatic or aromatic hydrocarbons which may optionally be halogenated, or in a mixture thereof.

Step b) is preferably carried out in C₁-C₄ alcohol, an aliphatic hydrocarbon or in a water/alcohol mixture. The reaction temperature is from 0 to 40° C., preferably from 5 to 30° C.

The ketone or aldehyde selected in step a) results in step b) in a nitrile compound of the formula (V) which crystallizes out of the reaction solution after addition of HCN.

The crystallized nitrile of the formula (V) is then where appropriate filtered off, washed and dried and converted in step c) by selective hydrolysis into the corresponding amide of the formula (VI).

Step b) and c) can also be carried out as one-pot reaction, in which case the nitrile is not isolated but directly hydrolyzed.

The selective hydrolysis takes place with use of a mineral acid such as, for instance, HCl, H₂SO₄, H₃PO₄. HCl is preferably used, and concentrated HCl is particularly preferably used.

The nitrile is in this case suspended in the mineral acid and stirred at a temperature of from 25 to 80° C., preferably from 35 to 60° C., for up to 15 hours.

The amide obtained in this way is in the form of a salt, for example hydrochloride, and is converted in step d) by use of an amidase or by use of a chiral resolving acid into the corresponding chiral amide. Examples of suitable amidase are L-amidase prepared from Mycobacterium neoaurum ATCC 25795, Mycobacterium smeginatis ATCC 19420 or Mycoplana dimorpha IFO 13291.

Suitable chiral resolving acids are, for example, the D and L forms of tartaric acid, dibenzoyltartaric acid, di-1,4-toluyltartaric acid, mandelic acid, p-bromo-mandelic acid, p-chloromandelic acid, p-methylmandelic acid, 10-camphorsulfonic acid, 3-bromocamphor-8-sulfonic acid, 3-bromocamphor-10-sulfonic acid, malic acid, 2-pyrrolidone-5-carboxylic acid, 2,3,4,6-di-O-isopropylidene-2-keto-L-gulonic acid, 2-(phenyl-carbamoyloxy)propionic acid, 2-phenoxypropionic acid, aspartic acid, N-benzoylaspartic acid, 2-(4-hydroxy-phenoxy)propionic acid, (4-chlorophenyl)-2-isopropyl-acetic acid, 2-(2,4-dichlorophenoxy)propionic acid, 2-hydroxy-4-phenylbutyric acid, 2-(4-chloro-2-methyl-phenoxy)propionic acid, N-benzoylglutamic acid, N-(p-nitrobenzoyl)glutamic acid, N-(p-chlorobenzoyl)glutamic acid, 3-phenyllactic acid or di-1,4-anisoyltartaric acid.

D- or L-tartaric acid or D- or L-di-1,4-toluyltartaric acid are preferably employed.

Finally, the chiral amide is converted by means of an acid such as, for instance, HCl or acetic acid or an HCl/acetic acid mixture into the desired chiral mercapto amino acid.

HCl is preferably used, and concentrated HCl is particularly preferably used.

The reaction in this case is preferably carried out under an inert nitrogen atmosphere at the reflux temperature.

However, it is also possible (step e) for the amide first to be reacted with the acid to give the corresponding (R,S)-mercapto amino acid, which is then converted by one of the abovementioned amidases or resolving acids into the corresponding chiral mercapto amino acid.

The desired final compound is isolated for example by extraction, crystallization etc., depending on the final compound.

The process of the invention results in the desired chiral mercapto amino acids in a simple, cost-effective manner in high yields and in high optical purity.

### EXAMPLE 1

### Preparation of spiro-2,2′-cyclohexyl-4-methylthiazoline (step a)

58 g (1034.6 mmol) of sodium hydrosulfide hydrate were suspended in 206 ml (1987.6 mmol) of cyclohexanone in a 500 ml reaction flask, and then diluted with 60 ml of water. Subsequently, at a temperature of from 0 to 5° C., 60 g (648.44 mmol) of chloroacetone and 134 ml (1789.0 mmol) of 25% strength ammonium hydroxide solution were slowly added dropwise simultaneously using a pump. The solution obtained in this way was stirred at 5 to 8° C. for 30 minutes.

The organic phase was separated off from the two-phase solution, and the thiazoline was isolated therefrom by distillation through a Vigreux column.

Yield of thiazoline of the formula (IV): 59.24 g (53.96%).

### EXAMPLE 2

### Step a) with Phase-Transfer Catalysis

Spiro-2,2′-cyclohexyl-4-methylthiazoline was prepared in analogy to example 1 but with phase-transfer catalysis.

When triethylmethylammonium chloride was used as phase-transfer catalyst, 33.59 g (70.34%) of thiazoline of the formula (IV) were obtained.

### EXAMPLE 3

### Step a) with Use of Solubilizers

96.6 g (1241 mmol) of sodium hydrosulfide hydrate were suspended in 342 ml (3300 mmol) of cyclohexanone and then dissolved by adding 223 ml of 25% strength ammonium hydroxide solution. 8.83 g of acetonitrile were added to this two-phase mixture and then, while cooling, 100 g (1081 mmol) of chloroacetone were slowly added dropwise at a temperature of 15 to 20° C. over the course of 2 hours. The reaction mixture was stirred at room temperature for 30 minutes. The aqueous phase was separated off from the two-phase solution. The thiazoline of the formula (IV) was isolated from the organic phase by rectification.

Yield of spiro-2,2′-cyclohexyl-4-methylthiazoline: 131.2 g (71.7% of theory).

### EXAMPLE 4

### Preparation of spiro-2,2′-cyclohexyl-5-dimethylthiazolin (step a)

109 g (1023 mmol) of alpha-chloroisobutyraldehyde were added dropwise to a suspension of 90.3 g (1160 mmol) of sodium hydrosulfide hydrate in 320 ml (3088 mmol) of cyclohexanone and 209 ml of 25% aqueous ammonia solution at 0° C. to 5° C. over the course of 1 h. The reaction mixture was stirred for 30 min. The aqueous phase was separated off. After removal of the cyclohexanone, the thiazoline was isolated by rectification.

Yield of spiro-2,2′-cyclohexyl-5-dimethylthiazoline: 108.8 g (58.9% of theory).

### EXAMPLE 5

### Preparation of spiro-2,2′-cyclohexanyl-4-methyl-3-thiazolidine-4-nitrile (step b)

25.01 g (147.7 mmol) of spiro-2,2′-cyclohexyl-4-methylthiazoline were dissolved in 25 ml of methanol and, while cooling at a temperature below 10° C., 15 ml (383 mmol) of hydrocyanic acid were metered in over the course of 10 min. After stirring for 30 min, the onset of crystallization was observed. After stirring at room temperature for 2 hours, 25 ml of water were added dropwise. The suspension was then stirred at room temperature for 30 min. Subsequently, the precipitate was filtered off and washed with cold 1:1 methanol/water. The white crystalline product was dried in vacuo at 40° C.

Yield of spiro-2,2′-cyclohexanyl-4-methyl-3-thiazolidine-4-nitrile: 25.05 g (90.0% of theory).

### EXAMPLE 6

### Preparation of 2,2-dimethyl-4-aza-1-thiaspiro[4,5]decane-3-carbonitrile (step b)

2,2-Dimethyl-4-aza-1-thiaspiro[4,5]decane-3-carbonitrile was prepared in analogy to example 5.

Yield of 2,2-dimethyl-4-aza-1-thiaspiro[4,5]decane-3-carbonitrile: 114.47 g (99.8% of theory).

### EXAMPLE 7

### Preparation of (R,S)-3-methyl-4-aza-1-thiaspiro[4,5]decane-3-carboxamide (step c)

5 g (25.47 mmol) of spiro-2,2′-cyclohexanyl-4-methyl-3-thiazolidine-4-nitrile were suspended in 63 ml of conc. hydrochloric acid and stirred at 45° C. for 2 hours. Then the suspension with white precipitate was cooled to about 5° C. and, after standing for a short time, filtered. The precipitate was washed 3× with cold water and 3× with cold methanol. It was then dried in vacuo at 35° C. overnight.

Yield of (R,S)-3-methyl-4-aza-1-thiaspiro[4,5]decane-3-carboxamide hydrochloride: 5.0 g (78.2% of theory).

The hydrochloride was suspended in 25 ml of water and adjusted to pH 8.6 with 25% strength ammonium hydroxide solution. The precipitate was filtered off and washed several times with cold water. The product was then dried in vacuo at 50° C.

Yield of (R,S)-3-methyl-4-aza-1-thiaspiro[4,5]decane-3-carboxamide: 4.0 g (73.9% of theory).

### EXAMPLE 8

### Preparation of (R,S)-3-methyl-4-aza-1-thiaspiro[4,5]decane-3-carboxamide in a one-pot process. (Step b+c)

3.0 g (17.7 mmol) of spiro-2,2′-cyclohexyl-4-methyl-thiazoline were dissolved in 7 ml of n-heptane, and 1.8 ml (44.3 mmol) of hydrocyanic acid were added. After stirring at room temperature for about 30 min, the resulting spiro-2,2′-cyclohexanyl-4-methyl-3-thiazolidine-4-nitrile was crystallized by cooling with ice-water. Subsequently, 30 ml of concentrated hydrochloric acid were added, and the suspension with white precipitate was stirred at 45 to 50° C. for 7 hours.

The pH was then adjusted to 8.5 by adding about 45 ml of 25% strength sodium hydroxide solution while cooling, and the precipitate was filtered off. The product was washed with water and dried in vacuo at 40° C.

Yield of (R,S)-3-methyl-4-aza-1-thiaspiro[4,5]decane-3-carboxamide: 2.2 g (58.3% of theory).

### EXAMPLE 9

### Preparation of 2,2-dimethyl-4-aza-1-thiaspiro[4,5]decane-3-carboxamide (step c)

A suspension of 10 g (32.2 mmol) of 2,2-dimethyl-4-aza-1-thiaspiro[4.5]decarb-3-carbonitrile in 100 ml of conc. hydrochloric acid was stirred at 58° C. to 60° C. for 10 h. After the reaction mixture had cooled, the hydrochloric acid was removed and the residue was mixed with 80 ml of water and 35 ml of toluene. The aqueous phase was adjusted to pH 9 with 25% aqueous ammonia solution. The product precipitated. The white solid was dissolved in 150 ml of hot water and 43 ml of methanol. Crystallization resulted in 5.27 g (48.6%) of amide.

### EXAMPLE 10

### Preparation of chiral 3-methyl-4-aza-1-thiaspiro[4,5]decane-3-carboxamide (step d)

21.1 g (140 mmol) of D-(−)-tartaric acid were dissolved in 180 ml of methanol and then 20 g (93.3 mmol) of (R,S)-3-methyl-4-aza-1-thiaspiro[4,5]decane-3-carboxamide were added. After stirring at room temperature for 15 minutes, the suspension was filtered and the precipitate was washed with methanol. Drying in vacuo at 50° C. resulted in 15.53 g (91.4% based on the desired enantiomer) of the diastereomeric salt were obtained with a chiral purity of 96.5%.

13.35 g of the diastereomeric salt were suspended in 133.5 ml of dist. water, and the pH was adjusted from about 3 to 8.35 by adding 6.5 ml of 25% ammonium hydroxide solution. The reaction mixture warmed by about 5° C. during this. After stirring at room temperature, the precipitate was filtered and washed three times with 15 ml of water. The free amide was dried in vacuo at 45° C. overnight. 7.02 g (89.4% based on the diastereomeric salt) of amide were isolated.

### EXAMPLE 11

### Preparation of Chiral Alpha-Methylcysteine

3.5 g of (R)-3-methyl-4-aza-1-thiaspiro[4,5]decane-3-carboxamide were suspended in 35 ml of conc. hydrochloric acid and slowly heated under an inert nitrogen atmosphere. There is initially much foaming of the suspension. For this reason, it was initially heated at 58° C. for 20 min, then at 70 to 80° C. for 45 min and finally to reflux. After about 7 h, the reaction solution was extracted with 12 ml of toluene. The aqueous phase was concentrated completely in a rotary evaporator, and the residue was dried with toluene. Subsequently, 30 ml of 2-butanol were added and digested at 56° C. The precipitate was filtered off and washed with 2-butanol. The filtrate was concentrated to a 25% solution. While cooling in ice, 150 ml of MtBE were slowly added dropwise. The precipitate was filtered off and dried at 56° C. 2.0 g (60%) of alpha-methylcysteine hydrochloride were obtained.

1. A process for preparing chiral mercapto amino acids of the formula 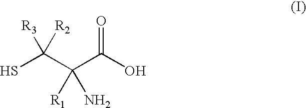
in which R₁, R₂ and R₃ may be identical or different and may be hydrogen, C₆-C₁₂-aryl, C₁-C₆-alkyl-C₆-C₁₂-aryl, C₆-C₁₂-aryl-C₁-C₆-alkyl, C₁-C₁₈-alkyl or C₂-C₁₈-alkenyl, where R₂ and R₃ may form a saturated or unsaturated ring, and the radicals may optionally be substituted one or more times by F, NO₂ or CN, characterized in that
a) an oxo compound of the formula 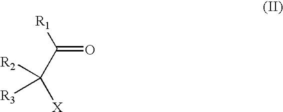
in which R₁, R₂ and R₃ are as defined above, and X is a leaving group from the group of Cl, Br, iodine, triflate, acetate or of the sulfonates, is reacted in the presence of ammonia or ammonium hydroxide and of a sulfide from the group of ammonium hydrosulfide, alkaline earth metal hydrosulfides or alkali metal hydrosulfides, where appropriate with phase-transfer catalysis or with addition of a solubilizer, with a ketone or aldehyde of the formula 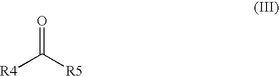
in which R₄ and R₅ may be identical or different and may be a C₁-C₁₂-alkyl radical or a C₆-C₂₀-aryl radical or one of the two radicals may be H, or R₄ and R₅ together form a C₄-C₇ ring which may optionally be substituted one or more times by C₁-C₆-alkyl or C₆-C₂₀-aryl, to give the compound of the formula 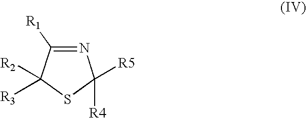
in which R₁, R₂, R₃, R₄ and R₅ are as defined above, which
b) reacts with HCN to give the compound of the formula 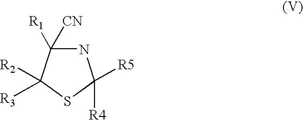
in which R₁, R₂, R₃, R₄ and R₅ are as defined above, after which
c) the crystallized compound of the formula (V) is converted by selective hydrolysis using a mineral acid into the corresponding amide of the formula 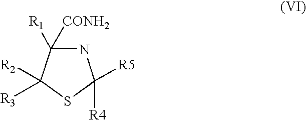
in which R₁, R₂, R₃, R₄ and R₅ are as defined above, and
d) subsequently converted using an amidase or a chiral resolving acid into the corresponding chiral amide of the formula (VI*), after which the desired chiral mercapto amino acid of the formula (I) is obtained by reaction with an acid, or
e) firstly the reaction of the amide with an acid is carried out, and subsequently the conversion into the desired chiral mercapto amino acid of the formula (I) takes place.

2. The process as claimed in claim 1, characterized in that in step a) from 1 to 5 mol of ketone or aldehyde of the formula (III), from 1 to 3 mol of sulfide compound and from 1 to 5 mol of ammonia or ammonium hydroxide are added per mol of oxo compound of the formula (II).

3. The process as claimed in claim 1, characterized in that in step a) a ketone of the formula (III) in which R₄ and R₅ together form a C₅-C₆ ring which may optionally be substituted one or more times by C₁-C₄-alkyl or phenyl is employed.

4. The process as claimed in claim 1, characterized in that in step b) HCN is employed as such, gaseous or liquid or as solution in water or organic solvents or prepared as intermediate from HCN and acid in an amount of from 1 to 5 mol per mol of thiazoline compound of the formula (IV).

5. The process as claimed in claim 1, characterized in that step b) is carried out in a solvent from the group of water, C₁-C₄-alcohol, ester, ether or optionally halogenated, aliphatic or aromatic hydrocarbons or mixtures thereof.

6. The process as claimed in claim 1, characterized in that in step c) the crystallized nitrile of the formula (V) is suspended in the mineral acid and stirred at from 25 to 80° C. for up to 15 hours, after which the amide of the formula (VI) is obtained as salt.

7. The process as claimed in claim 1, characterized in that step b) and c) take place as one-pot reaction, with the crystallized nitrile of the formula (V) not being isolated from the reaction mixture but being reacted immediately with the mineral acid to give the amide of the formula (VI).

8. The process as claimed in claim 1, characterized in that in step d) or e) an L-amidase prepared from Mycobacterium neoaurum ATCC 25795, Mycobacterium smeginatis ATCC 19420 or Mycoplana dimorpha IFO 13291 or a chiral resolving acid from the group of tartaric acid, dibenzoyltartaric acid, di-1,4-toluyltartaric acid, mandelic acid, p-bromomandelic acid, p-chloromandelic acid, p-tolytartaric acid, mandelic acid, p-bromomandelic acid, p-chloromandelic acid, p-methylmandelic acid, 10-camphorsulfonic acid, 3-bromocamphor-8-sulfonic acid, 3-bromocamphor-10-sulfonic acid, malic acid, 2-pyrrolidone-5-carboxylic acid, 2,3,4,6-di-O-isopropylidene-2-keto-L-gulonic acid, 2-(phenylcarbamoyloxy)propionic acid, 2-phenoxypropionic acid, aspartic acid, N-benzoylaspartic acid, 2-(4-hydroxy-phenoxy)propionic acid, (4-chlorophenyl)-2-isopropylacetic acid, 2-(2,4-dichlorophenoxy)propionic acid, 2-hydroxy-4-phenylbutyric acid, 2-(4-chloro-2-methyl-phenoxy)propionic acid, N-benzoylglutamic acid, N-(p-nitrobenzoyl)glutamic acid, N-(p-chlorobenzoyl)glutamic acid, 3-phenyllactic acid or di-1,4-anisoyltartaric acid in their D or L form is employed.

9. The process as claimed in claim 1, characterized in that the reaction with the acid in step d) and e) is carried out under an inert nitrogen atmosphere at the reflux temperature.
